(19) United States
(12) Patent Application Publication
(10) Pub. No.: US 2004/0102707 A1
(43) Pub. Date: May 27, 2004

(54) Acoustic coupler for medical imaging

(76) Inventor: John M. Murkin, London (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/645,572

(22) Filed: Aug. 22, 2003

Related U.S. Application Data
(60) Provisional application No. 60/404,914, filed on Aug. 22, 2002.

(51) Int. Cl. A61B 8/14

(52) U.S. Cl. 600/459

(57) ABSTRACT

An acoustic coupler for use with an ultrasound probe by a surgeon as a diagnostic tool is described. The present invention provides an acoustic coupler for use with an ultrasound probe for imaging an anatomical structure, comprising a member that is capable of being sterilized, is acoustically neutral, and is in vivo biocompatible, and comprises: (a) a first surface adapted to receive and fix the position of an ultrasound probe head relative to the member, to ensure the correct orientation of the probe head in relation to the anatomical structure during imaging; and (b) a second surface opposed to the first surface, the second surface being shaped to substantially conform to the contour of the anatomical structure. Also described is a method for producing ultrasonic images of an anatomical structure with an acoustic coupler and an ultrasound probe.

### FIELD OF THE INVENTION

The present invention relates generally to the field of diagnostic surgical tools, and more particularly relates to a device utilizing ultrasound for diagnostics.

### BACKGROUND OF THE INVENTION

### Stroke Risk After Cardiac Surgery

An estimated 330,000 surgical procedures were performed using cardiopulmonary bypass (CPB) in 1994 in the United States (Mills, 1995). With the Increasing age and incidence of concomitant disease, it is increasingly recognized that emboli from instrumentation of an atherosclerotic aorta is an important source of stroke and central nervous system (CNS) morbidity (Murkin et al., 1995: Blauth et al., 1992, and Tuman et al., 1992). There is a direct correlation between age, peripheral vascular disease, and insulin dependent diabetes mellitus (IDDM) and severe atherosclerosis of the ascending aorta and atheroemboli production (Blauth et al., 1992). In a large postmortem study of 221 patients dying after cardiac surgery, atheroemboli were present in the brains in 37% of patients with severe disease of the ascending aorta but only 22% of the patients without severe disease (Blauth et al., 1992). 95% of patients who had evidence of atheroemboli postmortem (and would have manifested all the signs of a stroke had they lived), had severe atherosclerosis of the ascending aorta (Sylviris et al., 1997). in a study of 2000 CAB patients, Tuman et at, (1992), reported an overall postoperative stroke rate of 2.8%, but in patients 65 to 74 it was 3.6% and in those over age 75 the stroke rate was 8.9%. Currently 30 to 40% of the population we operate upon for coronary bypass surgery is in this age range. Patients with a postoperative neurologic event had a ninefold increase in mortality (35.7% versus 4.0%).

### Current Detection of Aortic Plaque

In fewer than 50% of patients can the presence of aortic arch atheromatous disease be predicted preoperatively using chest X-ray (CXR), or aortogram (Hosoda et al., 1991). Furthermore, 50-80% of significant atherosclerotic lesions in the ascending aorta are missed by intra-operative palpation by the surgeon (Hosoda et al., 1991; Davila-Roman et al., 1994; Barzilai et al., 1989; Marschall et al., 1989; and Katz et al., 1992). Katz et al., (1992). found that in a prospective study involving 130 patients, 19 (83%) of 23 patients with severe disease as determined by transesophageal echocardiography (TEE) were graded normal or mild by palpation. While calcific aorta can be assessed reasonably well, “cheesy” atherosclerosis is extremely difficult to detect by palpation (Landymore and Kinley, 1983). Manual palpation of the aorta by the surgeon to assess for optimal cannulation sites is currently the standard of care in most cardiac surgical centers in North America Identifying severe aortic disease has important clinical implications because surgical technique, including aortic cannulation to connect to the heart-lung machine (cardiopulmonary bypass, CPB machine) and anastomosis of proximal coronary grafts, and other such interventions may be altered or relocated to avoid areas of atherosclerotic plaque and should reasonably result in a decrease in stroke fate and in mortality associated with patients undergoing cardiac surgery (Hosoda et al., 1991; Davila-Roman et al., 1994; Barzilai et al., 1989; Marschall et al., 1989; Katz et al., 1992; and Wareing et al., 1992)

### Intraoperative Aortic Scanning

Rather than manual palpation, intra-operative ultrasound studies of the aorta using transesophageal echocardiography (TEE) of the aorta has been recommended as a routine in order to detect aortic atherosclerosis and guide surgical cannulation etc (Hosoda et al., 1991). As opposed to the standard echocardiogram in which the probe is placed over the chest wall, in TEE the probe is passed into the esophagus (through the swallowing tube) and is positioned directly behind the heart. Once in the proper position, the probe bounces ultrasonic sound waves off of the heart and images of the cardiac structures are produced. However, (1) this is an expensive instrument (average $125,000-$500,000 capital cost), (2) it requires significant expertise and an independent dedicated operator (presence of a dedicated technician or specially trained physician) for its intraoperative usage, and (3) its ability to detect all aortic arch lesions has been questioned since the air-tissue interface resulting from the lung and trachea prevents the identification of lesions in the upper ascending aorta and the aortic arch, where cannulation is done (Seward et al., 1990; Konstadt et al., 1994; Sylvirs et al., 1992; and Kanchuger et al., 1994).

Alternatively, employment of a hand-held epiaortic B-mode scanning prone has been shown to be more efficacious than TEE and similarly alters the site of aortic cannulation and instrumentation in 20-24% of CPB cases (Barzilai et al., 1989; Ohteki et al., 1990; and Davila-Roman et al., 1991). Epiaortic B-mode scanning has been shown to be accurate in assessing severity and location of atherosclerosis of the ascending aorta and allowing modification of the standard technique for cannulation by choosing a safer site (Davila Roman et al., 1994and Wareing et al., 1992). Additionally, epiaortic scanning has been found to be more reliable in identifying plaque in the distal ascending aorta where TEE is less helpful. Katz and colleagues (1992) showed that all 5 patients in whom severe distal ascending plaque was found by direct epiaortic probe were missed by biplanar TEE. The use of this instrument would obviate the need for manual palpation of the aorta, in itself a cause of embolization (Karalis et al., 1992).

Despite the availability of the above technology, the standard of care continues to be visual inspection and palpation of the aorta by the surgeon. This is true even though visual inspection and palpation of the aorta identifies atheromatous disease in only 25-50% of patients, and even then underestimates atherosclerotic severity compared with ultrasound scanning (Seward et al., 1990; Konstadt et al., 1994; Sylviris et al, 1992; and Kanchuger et al., 1994).

### Disadvantages of the Prior Art Aortic Scanning Devices

Ultrasound is a diagnostic modality based on the interpretation of sound waves reflected off of various interfaces in anatomical structures The strengtn of the reflected sound waves from an interface back to the probe is directly proportional to the density differential between adjacent structures. Interfaces with high-density differentials, such as the air/tissue interface, reflect almost all of the sound back to the probe, preventing the imaging of deeper structures.

To overcome this problem, an acoustically neutral coupling mediais commonly used to eliminate the air/tissue interface. The coupling media is typically a viscous fluid or gel that is applied directly to the tissue being imaged. However, a viscous fluid or gel is inappropriate for us within the human body during surgery (intraoperative scanning). The surgeon does not want to introduce or leave behind any unnecessary material inside of the human body. Furthermore, the viscous fluid or gel is typically not in vivo biocompatible, and thus may inadvertently trigger an immune system response. Additionally, ensuring that the viscous fluid or gel is sterile can be difficult.

Other problems exist with flat probe surfaces that result in ineffective acoustic coupling between the probe and the tissue. For example, when an ultrasound probe is placed directly on a pulsating heart during intraoperative use, the probe is forced to move both horizontally and vertically, resulting in substandard imaging. The probe must remain still during imaging so that it can receive the reflected ultrasonic sound waves in its original orientation. Moreover, incomplete coverage and/or air pockets typically exist when a flat probe is placed directly onto an irregularly shaped organ- As a result, the image is often incomplete and/or distorted Furthermore, deformation of the organ often occurs when a flat probe is pressed firmly up against the organ being imaged. This often alters the image and/or the velocity of blood flow through the organ/artery.

Additionally, current scanning probes that are placed directly onto tissue often result in a loss of near field resolution. In aortic scanning, near field resolution is crucial for effectively detecting the locations of aortic plaque that lie on the walls of the aorta. Use of a coupling media can often enhance near field resolution.

There is a need for an acoustic coupler for use with an ultrasound probe that is capable of being sterilized, is acoustically neutral, and is in vivo biocompatible. Furthermore, the acoustic coupler must be shaped to conform to the contour of the anatomical structure being imaged, and must be designed to fix the ultrasound probe head in a position relative to the acoustic coupler that ensures an optimal orientation in relation to the anatomical structure.

### SUMMARY OF THE INVENTION

The present invention provides an acoustic coupler for use with an ultrasound probe for imaging an anatomical structure, comprising a member that is capable of being sterilized, is acoustically neutral, and is in vivo biocompatible, and comprises:

(a) a first surface adapted to receive and fix the position of an ultrasound probe head relative to the member, to ensure the correct orientation of the probe head in relation to the anatomical structure during imaging; and

(b) a second surface opposed to the first surface, the second surface being shaped to substantially conform to the contour of the anatomical structure.

In one embodiment of the invention, the member is a solid- In another embodiment of the invention, the member is at least a partially deformable semi-solid.

Preferably, the member is comprised of one or a combination of the following; gelatine, agar, and/or alginate.

In one embodiment of the invention, the second surface of the member has a concave groove and the anatomical structure is an artery. In another embodiment of the invention, the anatomical structure is an aorta.

In one embodiment, the acoustic coupler further comprises a sheath that is waterproof and capable of being sterilized, having a top end and a bottom end, wherein the top end of the sheath is adapted to provide a generally watertight closure and the bottom end is attached to the first surface of the member.

In one embodiment of the invention, the sheath is transparent and comprised of polyvinyl chloride. In another embodiment of the invention, the top end of the sheath includes a drawstring to provide the generally watertight closure.

The present invention provides an ultrasound probe assembly for imaging an anatomical structure, comprising:

(a) a probe head;

(b) an ultrasonic transducer housed by the probe head; and

(c) a member that is capable of being sterilized, is acoustically neutral and is in vivo biocompatible, comprising:

(i) a first surface adapted to receive and fix the position of the ultrasound probe head relative to the member, to ensure the correct orientation of the probe head in relation to the anatomical structure during imaging; and

(ii) a second surface opposed to the first surface, the second surface being shaped to substantially conform to the contour of the anatomical structure.

In one embodiment, the ultrasound probe assembly further comprises a sheath that is waterproof and capable of being sterilized, having a top end and a bottom end, wherein the top end of the sheath is adapted to provide a generally watertight closure and the bottom end is attached to the first surface of the member.

The present invention also provides a method of producing an ultrasonic image of an anatomical structure, comprising the steps of:

(a) providing an ultrasound probe head with a surface for transmitting and receiving ultrasonic energy;

(b) providing a member that is acoustically neutral and is in vivo biocompatible, comprising.

(i) a first surface having a depression to receive and fix the position of the ultrasound probe head relative to the member, to ensure the correct orientation of the probe head in relation to the anatomical structure during imaging; and

(ii) a second surface opposed to the first surface, The second surface being shaped to substantially conform to the contour of the anatomical structure;

(c) ensuring that the member at least is sterile;

(d) placing the probe head into the depression on the first surface of the member;

(e) placing the member onto the anatomical structure to be imaged: and

(f) transmitting and receiving ultrasonic energy to and/or from the anatomical structure through the member.

In on embodiment, the method further comprises providing a member with a sheath extending from the first surface of the member, and enclosing the ultrasound probe head in a sheath, to prevent contact of the ultrasound probe head with the patient.

In another embodiment, the method includes providing a plurality of members, wherein each member has a second surface which is at least partially a cylindrical surface and wherein the diameters of the cylindrical surfaces are different, and wherein the method includes selecting a member having a second surface providing the best match to the anatomical structure to be imaged.

### BRIEF DESCRIPTION OF THE DRAWINGS

For a better understanding of the present invention, and to show more clearly how it may be carried into effect, reference will now be made, by way of example, to the accompanying drawings, which show a preferred embodiment of the present invention and in which.

FIG. 1 is a simplified schematic cross-sectional view of an ultrasound probe;

FIG. 2 is a perspective view of an acoustic coupler according to the present invention;

FIG. 3a is a top plan view of a first surface of the acoustic coupler of FIG. 2;

FIG. 3b is a bottom plan view of a second surface on the acoustic coupler of FIG. 2: and

FIG. 4 is a perspective view of an ultrasound probe fitted into the acoustic coupler of FIG. 2.

### DETAILED DESCRIPTION OF THE INVENTION

Ultrasound is a diagnostic modality based on the interpretation of ultrasonic sound waves or signals reflected off of various interfaces in anatomical structures. The strength of the reflected signals from an interface back to the probe is directly proportional to the density differential between adjacent structures. Interfaces with high-density differentials, such as the air/tissue interface, reflect almost all of the signals back to the probe, preventing the imaging of deeper structures. To overcome this problem, an acoustically neutral coupling media is commonly used to eliminate the airitissue interface.

The ultrasound probe head houses a composite array of individual ultrasonic transducers adapted to transmit ultrasonic signals into living tissue and to receive reflected signals according to principles well known in the art The transducer functions alternately in “Doppler” mode or “B-mode”. In Doppler mode, flow velocity (FV) in a blood vessel is measured. In B-mode, the cross-sectional area (Area) of the blood vessel is measured. These determinations are standard, software derived parameters obtained from ultrasound scanning probes and are well known in the prior art. In summary, by calculating the product of mean flow velocity and cross-sectional area, flow in milliliters per second can be derived according to the equation:

FV(cm/sec)×Area(cm²)=Flow(cc/sec)

This will enable determination of the flow within the coronary artery and any other blood vessel being scanned to be readily determined by The operator.

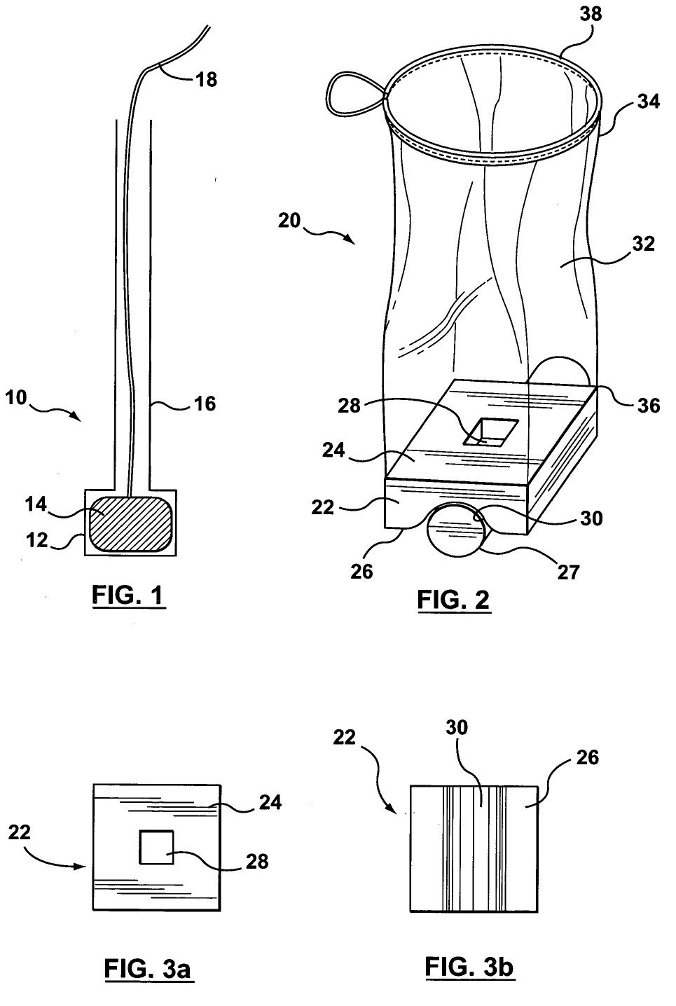

For simplicity, the preferred embodiment will refer to scanning of an aorta. However, it is to be understood that the present invention can be designed and used to scan any anatomical structure within the human body. For example, the present invention may be used to scan other types of blood vessels, including, but not limited to: carotid, renal, or hepatic arteries. In an alternative embodiment, the present invention may be used to scan organs, including, but not limited to: kidneys, livers, or brains. In an alternative embodiment, the present invention may be used transabdominally to scan the abdomen or chest prior to making a surgical incision. Referring first to FIG. 1, a schematic cross-sectional view of an ultrasound probe will be described. An ultrasound probe is shown generally at 10. The ultrasound probe 10 is comprised of an ultrasound probe head 12, an ultrasound transducer 14 that is housed in the probe head 12, and a handle 16 that is attached to the probe head 32. A wire 18 is attached to the transducer 14 for sending and receiving electronic signals.

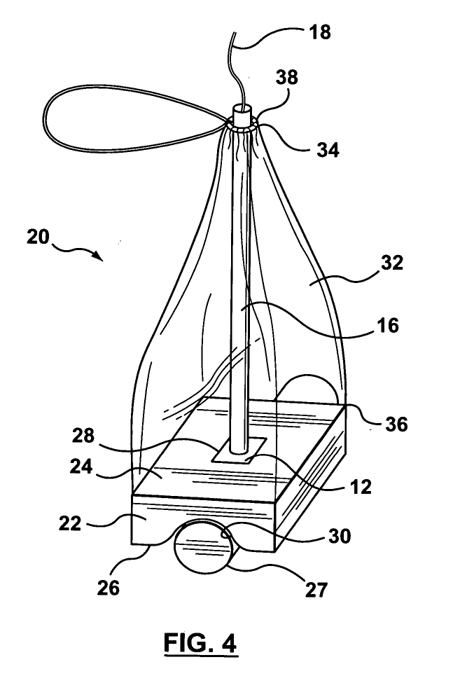

Referring to FIGS. 2 and 4, an acoustic coupler apparatus according to a first embodiment of the present invention is shown generally at 20. An acoustic coupler 22 comprises a member having a first surface 24 and a second surface 26. The first surface 24 of the acoustic coupler 22 has a depression 28 that is adapted to receive the ultrasound probe head 12 and fix its position relative to the acoustic coupler 22 for the duration of the scanning. This serves to ensure the correct orientation of the probe head in relation to the anatomical structure during imaging- The second surface 26 is generally shaped to conform to the contour of an aorta 27. More specifically, the second surface 26 contains a concave groove 30 that is shaped to fit over an aorta 27. Preferably, the acoustic coupler 22 has a flexible sheath 32 that is capable of being sterilized. The sheath 32 has a top end 34 and a bottom end 36. The top end 32 is preferably wrapped around a drawstring closure 38, which is used to provide a generally watertight closure around the probe handle 16 or wire 18, depending on the length of the sheath 32. Preferably, the bottom end 36 is molded to the first surface 24 of the acoustic coupler 22, thus providing an integral unit. The acoustic coupler 22 and sheath 32 act as a sterile barrier. It is understood that if a sterilized ultrasound probe 10 is used, the acoustic coupler 22 may be used alone without the sheath 32.

Referring now to FIG. 3a, a top plan view of the first surface 24 of the acoustic coupler 22 is shown. The depression 28 can be located anywhere on the first surface 24 of the acoustic coupler 22. The ultrasound probe head 12 is fitted into the depression 28 and remains relatively still during imaging. This ensures optimal orientation of the ultrasound probe head in relation to the aorta 27. The probe must remain still during imaging so that it can receive the reflected ultrasonic signals in its original orientation. This is especially relevant during intraoperative use when the aorta 27 being imaged is pulsating.

Referring now to FIG. 3b, a bottom plan view of the second surface 26 of the acoustic coupler 22 is shown. The second surface 26 is provided with the concave groove 30 that is shaped to fit directly onto the aorta 27, thus providing coverage of a substantial part of the organ. The concave groove 30 preferably is part of a cylindrical surface of constant diameter, equal to the diameter of the aorta 27. This concave groove 30 ensures that the ultrasound probe head 32 remains still relative to the aorta 27 during imaging, even though the aorta 27 is pulsating. Moreover, this shape prevents deformation of the aorta 27, which can often disturb the accuracy of the image and distort the measure of blood flow

The acoustic coupler 22 is capable of being sterile, is acoustically neutral, and is in vivo biocompatible material- For example, the acoustic coupler 22 may be comprised of one or more of the following materials: gelatine, agar, and/or alginate When the acoustic coupler 22 is being manufactured, its consistency can be controlled by the amount of water that is added to the mixture during boiling. Thus, the acoustic coupler 22 may be designed to be a semi-solid that is capable of at least partially deforming around the aorta 27. Alternatively, the acoustic coupler 22 may be designed to be substantially solid.

The acoustic coupler 22 comprising one or more of the materials mentioned above may be friable. Thus, the outer surface of the acoustic coupler 22 may optionally be laminated with a plastic film or the like. This ensures that the acoustic coupler 22 stays intact and does not degrade. In an alternative embodiment, the acoustic coupler 22 may be encased in a bag comprising plastic or the like.

In an alternative embodiment, the acoustic coupler 22 may comprise an enclosed bag filled with a material comprising a liquid phase acoustically inert saline solution.

The sheath 32 is comprised of a material that is preferably flexible, waterproof, and capable of being sterilized. For example, the sheath 32 may be comprised of polyvinyl chloride, or any other impermeable hypoallergenic plastic material well known in the art.

Now referring to FIG. 4, a perspective view of the ultrasound probe 10 fitted into the acoustic coupler and integral sheath 20 is shown. The optional drawstring closure 38 fits tightly around the ultrasound handle 16 or wire 18 to provide a generally watertight closure. This is important for maintaining sterility throughout the operation. The sheath 20 could be large enough so that the ultrasound handle 16 is completely enclosed. Then, the handle 16 could be gripped through the sheath 20.

The method of use of the acoustic coupler 10 is described below. Initially, the ultrasound probe 10 is brought down through the top end 34 of the sheath 32. Next, the ultrasound probe head 12 is fitted into the depression 28 located on the first surface 24 of the acoustic coupler 22. The drawstring 38 is then pull d closed to provide a generally watertight closure around the handle 16 of the ultrasound probe 10. Next, the second surface 26 of the acoustic coupler 22 is placed onto the aorta 27. In known manner, the ultrasound probe transmits and receives ultrasonic energy to and from the aorta 27 through the acoustic coupler 22.

While the present invention has been described with reference to what are presently considered to be the preferred embodiments, it is to be understood that the invention is not limited to the disclosed embodiments. To the contrary, the invention is intended to cover various modifications and equivalent arrangements Included within the spirit and scope of the appended claims. The acoustic coupler 22 could be used on a variety of arteries, in addition to use on the aorta 27. For example, the present invention may be used to scan other types of blood vessels, including, but not limited to: carotid, renal, hepatic or femoral arteries. Alternatively, with suitable reductions in the size of the concave groove 30, the acoustic coupler 22 may be used to image smaller blood vessels, including, but not limited to: coronary or cerebral arteries.

It will be understood that even where the acoustic coupler 22 is a semi-solid, the ability of the concave groove 30 to adapt to the arteries of different diameters is limited. Each artery can deflect to some extent, to give a large contact area with the concave groove 30- However, it is preferred to provide a number of acoustic couplers 22, each having a concave groove 30 of different diameter. Preferably, acoustic couplers 22 are provided with concave grooves having diameters in the range of between about 1 cm to 7 cm, and more preferably about 5 cm.

Preferably, the depression 28 is about 2 cm by about 3 cm, and has a depth of about 1.5 cm. The sheath 20 is preferably about 160 cm long, but all dimensions will be adapted to the dimensions of the ultrasound probe 10 used.

1. An acoustic coupler for use with an ultrasound probe for imaging an anatomical structure, comprising a member that is capable of being sterilized, is acoustically neutral, and is in vivo biocompatible, and comprises:
(a) a first surface adapted to receive and fix the position of an ultrasound probe head relative to the member, to ensure the correct orientation of the probe head in relation to the anatomical structure during imaging; and
(b) a second surface opposed to the first surface, the second surface being shaped to substantially conform to the contour of the anatomical structure.

2. An acoustic coupler according to claim 1, wherein the member is a Solid.

3. An acoustic coupler according to claim 1, wherein the member is at least a partially deformable semi-solid.

4. An acoustic coupler according to claims 2 or 3, wherein the member is comprised of gelatine.

5. An acoustic coupler according to claims 2 or 3, wherein the member is comprised of agar.

6. An acoustic coupler according to claims 2 or 3, wherein the member is comprised of alginate.

7. An acoustic coupler according to claims 2 or 3, wherein the member is comprised of a saline solution, and the solution is encased in a bag.

8. An acoustic coupler according to claim 1, wherein the second surface of the member has a concave groove and the anatomical structure is an artery.

9. An acoustic coupler according to claim 8, wherein the anatomical structure is an aorta.

10. An acoustic coupler according to claim 8, wherein the anatomical structure is an aortic arch.

11. An acoustic coupler according to claim 1, wherein the ultrasound coupler is adapted for intraoperative use.

12. An acoustic coupler according to claim 1, further comprising a sheath that is waterproof and capable of being sterilized, having a top end and a bottom end, wherein the top end of the sheath is adapted to provide a generally watertight closure and the bottom end is attached to the first surface of the member.

13. An acoustic coupler according to claim 12, wherein the sheath is transparent and comprised of polyvinyl chloride.

14. An acoustic coupler according to claim 12, wherein the top end of the sheath includes a drawstring to provide the generally watertight closure.

15. An ultrasound probe assembly for imaging an anatomical structure, comprising:
(a) a probe head;
(b) an ultrasonic transducer housed by the probe head; and
(c) a member that is capable of being sterilized, is acoustically neutral and in vivo biocompatible, comprising:
(i) a first surface adapted to receive and fix the position of the ultrasound probe head relative to the member, to ensure the correct orientation of the probe head in relation to the anatomical structure during imaging; and
(ii) a second surface opposed to the first surface, the second surface being shaped to substantially conform to the contour of the anatomical structure.

16. An ultrasound probe assembly according to claim 15, further comprising a sheath that is waterproof and capable of being sterilized, having a top end and a bottom end, wherein the top end of the sheath is adapted to provide a generally watertight closure and the bottom end is attached to the first surface of the member.

17. A method of producing an ultrasonic image of an anatomical structure, comprising the steps of:
(a) providing an ultrasound probe head with a surface for transmitting and receiving ultrasonic energy;
(b) providing a member that is acoustically neutral and in vivo biocompatible, comprising:
(i) a first surface having a depression to receive and fix the position of the ultrasound probe head relative to the member, to ensure the correct orientation of the probe head in relation to the anatomical structure during imaging; and
(ii) a second surface opposed to the first surface, the second surface being shaped to substantially conform to the contour of the anatomical structure,
(c) ensuring that the member at least is sterile;
(d) placing the probe head into the depression on the first surface of the member;
(e) placing the member onto the anatomical structure to be imaged; and
(f) transmitting and receiving ultrasonic energy to and/or from the anatomical structure through the member.

18. A method as claimed in claim 17, which includes providing a member with a sheath extending from the first surface of the member, and enclosing the ultrasound probe head in the sheath, to prevent contact of the ultrasound probe head with the patient.

19. A method as claimed in claim 17 or 18, which includes providing a plurality of members, wherein each member has a second surface which is at least partially a cylindrical surface and wherein the diameters of the cylindrical surfaces are different, and wherein the method includes selecting a member having a second surface providing the test match to the anatomical structure to be imaged.
